(19) United States
(12) Patent Application Publication
(10) Pub. No.: US 2009/0061016 A1
(43) Pub. Date: Mar. 5, 2009

(54) Seawater Based Dietary Supplement Product for Energy and Electrolyte Replacement

(76) Inventors: Jonathan A. Selzer, Cheshire, CT (US); Leah R. Haibi, Canton, CT (US)

Correspondence Address: ARTHUR G. SCHAIER;CARMODY & TORRANCE LLP, 50 LEAVENWORTH STREET, P.O. BOX 1110, WATERBURY, CT, 06721, US

(21) Appl. No.: 12/194,692

(22) Filed: Aug. 20, 2008

Related U.S. Application Data
(63) Continuation-in-part of application No. 11/899,263, filed on Sep. 5, 2007.

(51) Int. Cl. A61K 33/00; A61K 36/17; A61K 36/00

(52) U.S. Cl. 424/600; 424/729; 424/725

(57) ABSTRACT

A seawater based dietary supplement or functional food beverage product is revealed with enhanced energy profile, antioxidant properties, flavor and health characteristics. Seawater sources are combined with extracts of green or white tea, grape seed extract, d-ribose, extract of the roots or rhizomes of Eleuthero, extracts of the fruit of Lo Han and/or extracts from the leaves of Stevia and/or extracts from the leaves of Chinese Blackberry and/or extracts from the Sevilla orange. Green coffee extract or cocoa extract can be added to improve the antioxidant profile of the product. The resulting product is a pleasant tasting dietary supplement, which is easily absorbed by the body in liquid form, and provides substantial therapeutic effects.

This application is a continuation in part of U.S. patent application Ser. No. 11/899,263, filed on Sep. 5, 2007, which is currently pending.

### FIELD OF THE INVENTION

The present invention relates to seawater based compositions with improved antioxidant content, energy and electrolyte replacement and to a method for using such compositions as dietary supplements and/or therapeutic supplements and/or sweet functional food beverage products. The electrolytes in this invention are from seawater extracts; the energy is provided by D-ribose and the antioxidant properties are provided by herbal extracts with antioxidant activity, such as green tea extract, grape seed extract and Eleuthero root extract. Functional foods are defined as any conventional food that has been fortified to improve its nutritional value or is claimed to have health-promoting benefits above and beyond its normal nutritional value.

### BACKGROUND OF THE INVENTION

The uses of seawater as a therapeutic supplement are manifold and well documented. Animal life began in the sea and seawater closely matches the salt composition in the vertebrate body.

Although all organisms contain water, it is not the amount of water that is critical to maintain life, but the water concentration, or chemical activity that controls the dissolved ions. Since these dissolved ions regulate almost all cellular reactions and metabolism in the body, it is important to maintain relatively constant concentrations. Animal bodies expend considerable amounts of energy to maintain the ionic balance, otherwise known as osmotic equilibrium. Humans are osmoregulators, and maintain constant internal concentrations regardless of the conditions of their external environment. The mechanisms used to maintain homeostasis include the senses, coordination of neurons, hormones and behavior.

Mammals can be subjected to osmotic stress by dehydration or excessive water intake. Dehydration in humans occurs via the urine, feces, sweat, lungs and insensible evaporation over the skin. The kidneys are the main organ for maintaining human osmotic equilibrium. A resting man of 70 kg could lose up to 3.5 liters of water per day, or 5% of total body weight due to these factors. Exercise significantly increases the loss of water via sweat and evaporation over the lungs. Along with sweat, we lose a significant amount of salts. Together, the result is an imbalance of water and salt content in the blood. If this osmotic stress is not corrected, severe damage and death can occur. The kidneys, which work to compensate for unbalanced blood, are often the first to succumb to the added stress.

Humans compensate for dehydration by drinking water and eating food that contains water. However, dangerous conditions can also occur if much salt has been lost and a person drinks pure water. This can lead to low salt concentrations in the blood with equally severe consequences. In either condition, the kidneys must work hard to return the blood to osmotic equilibrium. This process can be aided by ingestion of salt solutions, which help to bring the blood back to osmotic equilibrium. The best solution is a physiologically meaningful solution of mineral ions in a form that can be absorbed and utilized by the body. Since seawater has a composition very close to that of the human body, it makes a very good source of electrolytes to return blood to osmotic equilibrium.

It has been well documented that exercise increases the amount of ROS (reactive oxidizing species) in the body. These ROS, which include the well-known free-radicals, can cause damage to tissue and cells in the body. This can cause increased risk of any number of oxidation-related diseases, including cardiovascular disease, stroke, cancer and accelerated aging.

For this reason, powerful antioxidants have been added to the invention, which is designed for people to use during and immediately after exercise. Green tea polyphenols are excellent sources of antioxidants for this invention. They are well known antioxidants, which have been extensively studied in the scientific literature. The polyphenols in green tea are water soluble and are easily combined with the other ingredients of this invention.

The most well documented therapeutic effect of green tea concerns the antioxidant properties of it's components called polyphenols and in particular the subgroup called catechins. The powerful antioxidant properties of the catechins is what is responsible for most of the health benefits, including the prevention of oxidation of LDL cholesterol vasorelaxation, anti-cancer properties and more.

Furthermore, green tea has other health benefits, not necessarily related to its antioxidant properties. These include the improvement of bone density, improved kidney health, improved liver health, weight loss and more.

Even before the appearance of scientific literature, many people have been consuming green tea products as either therapeutic herbal supplements or a refreshing drink or both. Green tea is one of the oldest drinks from countries such as Japan and China, where its health benefits have been revered for centuries.

The green tea used in conventional products, such as tea bags and ready made beverages are of varying qualities. Other components of green tea, which are not so desirable, can include fluoride and vitamin K1. Higher polyphenol content dictates a greater bitterness to the product. Drinks that contain sufficient polyphenols content to achieve beneficial effects can be objectionable because of the intense bitterness of the catechins.

Other antioxidant botanical extracts serve as alternatives to green tea extract, where taste, therapeutic benefits or intrinsic product stability dictate the absence of green tea. These can include grape seed extract and or Eleuthero root extract. Much scientific literature is available on the antioxidant properties of grape seed extract as well as its other health benefits.

The antioxidant properties of grape seed extract is due primarily to its proanthocyanidins content. Also a type of polyphenol, these exert biological properties similar to those of green tea extract. Grape seed extracts containing 95% polyphenols are easily available on the market today.

Although not as much well known in the United States, the diglycerides (triterpenoid saponins) from Eleuthero senticosus are active antioxidants. Formerly marketed in the United States as Siberian Ginseng, Eleuthero has many of the same health benefits as its more well-known relative, Ginseng (Panax ginseng). The taste profile of Eleuthero is much more pleasant than that of Ginseng and is therefore better suited to the invention presented herein.

Eleuthero has other properties, which make it appropriate for use in this invention. It has long been used in traditional Chinese medicine as an adaptagens. These are botanical substances, which help the body resist stress, fatigue, anxiety and trauma. All these properties make Eleuthero an invaluable component of an energy/sports beverage. Other adaptagens suitable to use in this invention include Licorice root, Ginseng root, Rhodiola root, Schisandra berry, Cordyceps and Reishi mushroom.

The third component of this invention is a source of energy for the athlete to utilize during periods of exercise and thereafter to regain energy expended during the exercise. One of the molecular bases of energy in mammals is d-ribose, a 5-carbon sugar that is required for the synthesis of energy in the form of ATP (adenosine triphosphate). Ribose is not plentiful in the human diet and we must synthesize it in our cells. This is a slow process and severely limits the rate at which ATP can be formed.

Metabolic stress is a condition of low energy stores that have been depleted by exercise, strenuous work, drug interactions, disease, etc. Without sufficient amounts of energy, the body's metabolic processes slow or cease altogether. This can have devastating effects on the body. Less life-threatening results of low energy include increased recovery times for athletes after exercise.

Ribose increases the rate of energy recovery and helps to build up energy stores. This is essential not only for the athlete, but also for all people at all times. Athletes have been shown to recover energy supplies more quickly, if taking ribose. People with diseases that interfere with energy production, such as chronic myalgia and myoadenylate deaminase can benefit from taking ribose to improve their ability to synthesize energy.

Furthermore, ribose is a non-glycemic sugar, which does not cause spikes in blood glucose levels. Ribose is utilized differently than other sugars, such as sucrose (regular table sugar), fructose or glucose. Thus ribose is suitable for diabetics and those involved with weight loss.

Additionally, the proposed therapeutic supplement will contain an extract of Eleuthero root and/or rhizomes. The adaptagenic Eleuthero extract is useful in this application to provide energy, improve circulation, resist stress and fatigue, and strengthen ligaments and bones. Eleuthero extract is also useful in this application for improving liver and kidney health.

Finally, since liquid dietary supplements are beverages, which must be taken through the mouth, taste is of the essence. The inventors have discovered that extracts derived from the fruits of Lo Han, extracts derived from the leaves of Stevia, extracts derived from the fruit of Citrus aurantium (also know an Seville orange) and extracts derived from the leaves of Chinese Blackberry are extremely effective taste improvers, adding flavor and sweetness to the invention.

Thus, it is an object of this invention to reveal a seawater based composition with sufficient ribose, green tea polyphenol concentration, and/or grape seed polyphenol and/or Eleuthero root and/or rhizome extract such that it provides substantial beneficial and therapeutic effects when consumed yet has a pleasant flavor. It is a further object to this invention to enhance the therapeutic benefits of seawater, ribose and green tea polyphenols by combining them with flavor enhancing additives, which themselves have beneficial therapeutic properties but which do not have the problems associated with the use of sugar.

### SUMMARY OF THE INVENTION

The inventors herein propose combining (i) seawater extract, (ii) d-ribose, (iii) Eleuthero (Eleuthero senticosus) root and/or rhizome extract, (iv) green tea (Camellia sinesis) extract and/or grape seed extract with (v) one or more additives selected from the group consisting of extracts derived from the fruits of Lo Han (i.e. Luo Han), extracts derived from the leaves of Stevia, and/or, extracts derived from the leaves of Chinese Blackberry, and extracts derived from the fruit of the Seville Orange, to produce an aqueous drink. The inventors have discovered that dietary supplements prepared in accordance with the foregoing, produce a great tasting herbal product which possesses many beneficial therapeutic properties, including the ability to boost the body's antioxidant levels, increase resistance to fatigue and stress, recover electrolyte balance after sweating and improve muscle recovery after exercise.

### DETAILED DESCRIPTION OF THE INVENTION

The inventors herein propose a dietary supplement and/or beverage product comprising:
(a) one or more of (i) green tea extract, (ii) white tea extract, (iii) extract of Camellia Sinesis, all preferably with polyphenol contents of between 10% and 98% by weight, or
   grape seed extract preferably with polyphenol contents of between 10% and 95% by weight, or
   Eleuthero root or rhizome extract preferably with eleutheroside content of between 0.1% and 25%; and
(b) d-ribose powder;
(c) seawater derived additive comprising at least one material selected from the group consisting of inorganic salts derived (extracted) from seawater, minerals derived (extracted) from seawater and combinations of the foregoing;
(d) at least one additive selected from the group consisting of (i) extracts of the fruits of Lo Han, (ii) extracts from the leaves of Stevia, (iii) extracts from the leaves of Chinese Blackberry, and (iv) extracts from the fruits of the Seville orange.
   Additional optional ingredients include xylitol, fructose or other preferably low glycemic sugars, coffee bean extract, vitamins, minerals, other herbal extracts, stabilizers such as Chinese licorice root extracts, and thickeners or gelling agents. The combination of the foregoing ingredients provides a great tasting dietary supplement or sweet functional food beverage product which possesses the therapeutic properties provided for herein.

The dietary supplement or functional food beverage product described herein requires a source of electrolyte minerals. The inventors have found seawater extract to be a useful source of electrolyte minerals for use in this invention. Seawater extract is preferred because of its concentrated form composition that greatly matches that of minerals in the human body. In choosing a source of seawater extract, it is preferred that the source of seawater not be subjected to pollution and other contaminants. Seawater extracts with reduced sodium contents are also preferred because they can be taken by individuals, who must limit their sodium intake for health reasons. The concentration of seawater extract in the concentrated dietary supplement, on a dry basis, may range from about 1% to 55% by weight. In the functional food beverage product, the concentration of seawater extract, on a dry basis, is preferably from about 0.1% to 10% by weight. One such source of seawater extract is ATOLIGOMER® from Codif, a company in France. This product comprises a seawater concentrate, from which sodium has been partially removed, and is subjected to a spray drying process. What is preferred is that the seawater derived additive comprise a mixture of inorganic salts and minerals derived or concentrated from seawater in approximately the same relative proportion found in natural seawater except that preferably a portion of the sodium is removed. The sea water extract can be prepared by concentrating sea water through evaporation, osmosis, use of semi-permeable membranes or other similar concentrating or extraction technology.

The dietary supplement or functional beverage product described herein preferably contains a source of antioxidants such as a source of green tea or white tea or a source of grape seed extract. The inventors have found green tea extract or white tea extract to be useful sources of polyphenols for use in this invention. In choosing a source of green tea or white tea, it is preferred that the polyphenol content ranges from about 50% to 98% by weight in the extract. The concentration of green tea, white tea, or grape seed extract in the dietary supplement, functional beverage on a dry basis may range from about 0.1% to 15% by weight. One suitable source of the green tea extracts or white tea extracts is Lycome Pharmaceuticals in Shanxii, China. If grape seed extract is used, preferably the extract contains from 10-95% by weight polyphenols. The green tea, white tea, or grape seed extracts are preferably aqueous and/or alcoholic extracts of the source.

The dietary supplement or functional beverage product described herein preferably requires a source of d-ribose. The inventors have found Bioenergy D-Ribose, from Valen Labs Inc. in Minneapolis Minn. for use in this invention. The concentration of d-ribose in the dietary supplement, functional beverage on a dry basis may range from about 1% to 50% by weight.

The dietary supplement or functional beverage product described herein preferably contains an extract of the roots and/or rhizomes of Eleuthero. Eleuthero is a member of the Araliaceae family and is also known as Siberian Ginseng or Ussurian Thorny Pepperbush. It is a plant native to the Taiga region of southeastern Russia, northern China, Korea and Japan. Extracts of the roots and/or rhizomes of Eleuthero are beneficial in (i) stimulating energy production, (ii) improving circulation, (iii) strengthening ligaments and bones, (iv) improving liver and kidney function and fighting respiratory tract infections. The Eleuthero extract useful in this invention can be made by contacting the chopped or crushed roots and/or rhizomes of Eleuthero plant with a water or water alcohol bath, preferably at elevated temperature for a period of time. The methods described herein for preparation of Lohan extracts are useful in preparing Eleuthero extracts. The concentration of Eleuthero extract in the product should be such that one recommended dose of the product provides the user with between about 20 mg and 200 mg of Eleuthero extract on a dry basis. A useful commercial extract of Eleuthero is available from the Naturex Company of South Hackensack, N.J.

The dietary supplement or functional food beverage product described herein also preferably contains at least one additive selected from the group consisting of (i) extracts of the fruits of Lo Han, (ii) extracts from the leaves of Stevia, (iii) extracts from the leaves of Chinese Blackberry, and (iv) extracts from the fruits of the Seville orange. Preferably more than one or all of the foregoing additives are present in the composition. The amount of the foregoing additives in the composition may range in concentration, depending upon the concentration of the extracts themselves. Generally, these extracts are provided as dried powders and if so, the concentration of these additives in the composition of this invention will range from about 0.05% to 6% by weight but is preferably from about 0.05% to 1% by weight.

Lo Han fruit comes from Momordica grosvenorii also called Siraitia grosvenorii. Lo Han is native to the People's Republic of China and Japan and is an edible fruit having an intensely sweet taste. It is reputed to possess healing properties for lung congestion, colds, sore throats, digestive and urinary disturbances, as well as antibiotic and antiseptic properties. Extracts of both fresh and dried Lo Han are available throughout the world, and can be readily purchased. One suitable aqueous extract is available from the Benyo Phytochemical Company, located in China. Various methods are known for preparing extracts of the Lo Han fruit, and other natural sources, such as those methods described in the U.S. Pat. Nos. 6,124,442 and 4,084,010, the teachings each of which are incorporated herein by reference in their entirety.

The preferred extracts of Lo Han are prepared as noted above using water or water/alcohol mixtures to extract the active species from the plant, thereby creating an aqueous extract. One such preferred method is described in U.S. Pat. No. 6,124,442, wherein the starting plant source material is fresh plant material such as cut pieces of freshly harvested Lo Han fruit. These fresh plant pieces are extracted by soaking in a bath of heated (80° F.-212° F.) water, alcohol (preferably ethanol) or both. This initial process is preferably carried out in hot, preferably boiling water or water/alcohol mixture. Extraction in the bath may be carried out several times, each time saving the resulting liquid. The resulting extract liquid is then filtered through a suitable filter, such as a 40-mesh stainless steel screen. The thus-obtained solid material can then be extracted again. It may be preferable for a particular process to carry out more or less extraction steps with different volumes of liquid being used for each boiling or extraction step. The result of this extraction process is an aqueous, alcohol or aqueous/alcohol extract that can be used in the composition of this invention. The extract can be used directly or concentrated by evaporation or spray drying and then used. Other extracts herein such as extract of the leaves of Stevia, extracts of the leaves of Chinese Blackberry or extracts of green or white tea (Camellia Sinesis) or extracts of grape seed, or extracts of Eleuthero or extracts of Seville orange may be made in the same way. If alcohol is used, alone or with water, it is preferably ethanol.

Stevia rebaudiana (Stevia) is also a plant which is native to the People's Republic of China. Extracts from the leaves of Stevia are commonly known and have been used as a natural sweetener in Asia for many years. Studies have demonstrated that extracts from the leaves of Stevia can have a variety of beneficial therapeutic effects including antibiotic properties especially against E. coli, vasodilatation properties, especially in the kidneys, anti-hypertensive effects, beneficial effects on pancreatic beta cells, and enhancing the secretion of insulin. Extracts from the leaves of Stevia are known and can be purchased for use in formulating the compositions described herein. These extracts can be prepared using the same or similar techniques used for preparing extracts for Lo Han as noted above. One suitable aqueous extract is available from the Benyo Phytochemical Company located in China.

Extracts from the leaves of Chinese Blackberry (Rubus suavissimus) are also known and can be purchased for use in formulating the compositions described herein. One suitable aqueous extract is available from the Benyo Phytochemical Company located in China. The active ingredients in these extracts are believed to comprise diterpene glycosides. Extracts of the leaves of Chinese Blackberry have been used as natural sweeteners. A variety of therapeutic benefits have been ascribed to extracts of the leaves of Chinese Blackberry, including relief from pre-menstrual syndrome, beneficial effects upon the kidney, and other beneficial effects. Extracts of the leaves of Chinese Blackberry can be prepared using the same or similar techniques used for preparing extracts of Lo Han, as noted above.

Extracts from the fruits of Seville orange (Citrus aurantium) are also known and can be purchased for use in formulating the compositions described herein. One suitable aqueous extract is available from the Benyo Phytochemical Company located in China. The active ingredients in these extracts are believed to comprise naringin dihydrochalone. Extracts of the fruits of Seville orange have been used as natural sweeteners. A variety of therapeutic benefits have been ascribed to extracts of the fruits of the Seville orange, including increased energy and other beneficial effects. Extracts of the fruits of the Seville orange can be prepared using the same or similar techniques used for preparing extracts of Lo Han, as noted above.

As noted, other optional materials may be included in the composition of this invention. Coffee bean extract is a powerful antioxidant, containing high levels of polyphenols. Cocoa bean extract is also a powerful antioxidant and can be used in the same manner as coffee bean extract. These herbal extracts are very effective at improving the antioxidant level of the seawater product and are suggested additives. Vitamins, minerals or other herbal extracts may be added for particular purposes or effects. Sugars such as fructose may be added but are not recommended. Artificial sweeteners or polyols, such as xylitol may be added and may be beneficial in formulating the compositions of this invention. Preferably the composition is substantially free of sucrose. Other flavoring agents such as natural orange flavor or natural berry flavor may also be utilized. Glycerin has also proven to be a beneficial additive.

Coffee bean extract is available from Futureceuticals, a supplier of extracts to the dietary supplement industry. Cocoa bean extract is available from Naturex Botanicals, a French company.

In preparing the compositions of this invention for use as dietary supplements, the ingredients noted herein are merely combined with water in the concentrations noted to form a dietary supplement drink. It is recommended that the consumer of the product ingest one to two sixteen-ounce servings of the diluted product per day, such that approximately 800 mg of d-ribose and 500 mg of seawater extract on a dry basis, are ingested per day. In preparing the compositions described herein as a functional food beverage, the ingredients taught herein are combined with larger quantities of water and appropriate stabilizers.

The compositions of this invention are further described in the following examples which should be taken as illustrative only and not limiting in any manner.

### EXAMPLE I

A cocoa based dietary supplement was prepared using the following ingredients:

| Component | % by Weight |
|---|---|
| Lo Han extract powder¹ | 0.5 |
| Seawater extract² | 1.0 |
| White tea extract³ | 1.0 |
| Natural orange flavor⁴ | 2.5 |
| d-ribose powder⁵ | 30.0 |
| Glycerin | 15.0 |
| Eleuthero Extract⁶ | 3.5 |
| Water | qsp |

¹Available from Benyo Phytochemical Company
²Available from the Codif Company
³Available from Lycome Industries, Shanxii, China
⁴Available from the Virginia Dare Company
⁵Available from Valen Labs
⁶Available from Naturex

The foregoing ingredients were blended to form a pleasant tasting dietary supplement which is believed to exhibit the beneficial therapeutic effects noted herein.

### EXAMPLE II

Example I was repeated except that Stevia extract powder (available from Benyo Phytochemical Company) was substituted for the Lo. Han extract powder at the same concentration. The same results were achieved.

### EXAMPLE III

Example I was repeated except that Chinese Blackberry extract powder (available from Shaanxi Company) was substituted for the Lo Han extract powder at the same concentrations. The same results were achieved.

### EXAMPLE IV

A seawater based dietary supplement was prepared using the following ingredients:

| Component | % by Weight |
|---|---|
| Seawater extract | 1.0 |
| Lo Han extract powder | 1.5 |
| Green tea extract powder | 3.0 |
| Eleuthero extract | 3.5 |
| D-ribose powder | 30 |
| Natural cocoa flavor | 14 |
| Water | qsp |

The foregoing ingredients were blended to form a pleasant tasting dietary supplement which is believed to exhibit the beneficial therapeutic effects noted herein.

### EXAMPLE V

A seawater based functional food beverage was prepared using the following ingredients:

| Component | % by Weight |
|---|---|
| d-ribose | 5.0 |
| seawater extract | 1.0 |
| Green tea extract | 2.0 |
| Lo Han extract powder | 1.5 |
| Eleuthero Extract | 3.5 |
| Water | qsp |

The foregoing ingredients were blended and pasteurized to form a functional food beverage.

What is claimed is:

1. A composition useful as a dietary supplement or a functional food beverage product, which composition comprises:
(a) a source of at least one material selected from the group of inorganic salts derived from seawater, minerals derived from seawater and combination of the foregoing; and
(b) at least one sweetener selected from the group consisting of (i) extracts of the fruits of Lo Han, (ii) extracts of the leaves of Stevia, (iii) extracts of the leaves of Chinese Blackberry and (iv) extracts of the Seville orange; and
(c) a source of antioxidants selected from the group consisting of green tea extracts, white tea extracts, grape seed extracts, Eleuthero root or rhizome extracts and combinations of the foregoing; and
(d) d-ribose.

2. A composition according to claim 1 wherein the composition also comprises at least one material selected from the group consisting of green coffee extract, tea extract, vitamins, fructose, glycerin, thickeners, gelling agents, xyitol and citric acid.

3. A composition according to claim 1 wherein the composition is substantially free of sucrose.

4. A composition according to claim 1 wherein the concentration of the source of the at least one material, on a dry basis, is from about 0.1% to 55% by weight.

5. A composition according to claim 1 wherein the concentration of the source of green tea, white tea extract or grape seed extract on a dry basis, is from about 0.1% to 15% by weight.

6. A composition according to claim 1 wherein the concentration of the source of d-ribose on a dry basis, is from about 0.1% to 50% by weight.

7. A composition according to claim 1 wherein the concentration of Eleuthero extract is such that from about 20 mg to 200 mg, on a dry basis, are provided to the user in one serving.

8. A composition according to claim 2 wherein the concentration of the inorganic salts derived from sea water and minerals derived from sea water together on a dry basis, is from about 0.1% to 55% by weight.

9. A composition according to claim 2 wherein the concentration of the green tea or white tea extract or grape seed extract, on a dry basis, is from about 0.1% to 15% by weight.

10. A composition according to claim 2 wherein the concentration of the d-ribose on a dry basis, is from about 0.1% to 50% by weight.

11. A composition according to claim 2 wherein the concentration of the material(s), on a dry basis, is from about 0.05% to 5.0% by weight.

12. A composition according to claim 1 wherein the source of at least one material comprises a natural seawater concentrate.

13. A composition useful as a dietary supplement or a functional food beverage product, which composition comprises;
(a) concentrated seawater;
(b) at least one sweetener selected from the group consisting of (i) extracts of the fruits of Lo Han, (ii) extracts of the leaves of Stevia, (iii) extracts of the leaves of Chinese Blackberry, and (iv) extracts of the fruit of the Seville orange;
(c) a source of antioxidants selected from the group consisting of green tea extracts, white tea extracts, grape seed extract, Eleuthero root or rhizome extract and combinations of the foregoing; and
(d) d-ribose.

14. A composition according to claim 13 wherein the composition comprises Eleuthero extract.

15. A composition according to claim 14 wherein the composition comprises at least one of (i) green tea extract, (ii) white tea extract, and (iii) grape seed extract.

16. A composition according to claim 14 wherein the concentration of d-ribose is from about 0.1% to 50% by weight.

17. A composition according to claim 14 wherein the concentration of Eleuthero extract is such that from about 20 mg to 200 mg, on a day basis, are provided to the user in one serving.

18. A composition according to claim 14, wherein the composition also comprises at least one material selected from the group consisting of green coffee extract, vitamins, fructose, glycerine, thickness, gelling agents, xylitol and citric acid.
